(19) United States
(12) Patent Application Publication
(10) Pub. No.: US 2026/0265180 A1
(43) Pub. Date: Sep. 10, 2026

(54) HALOGENATED BENZYLIDINE DERIVATIVES AND THEIR USE IN THE TREATMENT OF CANCERS

(71) Applicant: Renovel Innovations, Inc., Fremont, CA (US)

(72) Inventors: Bishwajit NAG, Union City, CA (US); Ananda Sen, Castro Valley, CA (US); Nitish Nag, Union City, CA (US); Arjun Sanyal, Castro Valley, CA (US); Srinivasan Narasimhan, Chennai (IN)

(73) Assignee: Renovel Innovations, Inc., Fremont, CA (US)

(21) Appl. No.: 19/660,850

(22) Filed: Apr. 28, 2026

Related U.S. Application Data
(63) Continuation-in-part of application No. 19/275,436, filed on Jul. 21, 2025, now Pat. No. 12,637,416.
Application No. 19/275,436 is a continuation-in-part of application No. 18/942,833, filed on Nov. 11, 2024.
Application No. 18/942,833 is a continuation of application No. 17/584,189, filed on Jan. 25, 2022, now Pat. No. 12,195,418.
(60) Provisional application No. 63/141,880, filed on Jan. 26, 2021.

(51) Int. Cl. C07C 251/24; C07C 229/36; C07D 213/74; C07D 213/80

(52) U.S. Cl. CPC C07C 251/24; C07C 229/36; C07D 213/74; C07D 213/80

(57) ABSTRACT

The present invention relates to novel halogenated benzylidene derivatives for the treatment of cancers,

### RELATIONSHIP TO OTHER APPLICATIONS

This application is a continuation-in part of U.S. Pat. No. 19,275,436 (pending) filed 21 Jul. 2025, which is a continuation-in part of 18942833 that was filed 11 Nov. 2024, and which is a continuation of U.S. Pat. No. 17,584,189, filed on 25 Jan. 2022, which was patented as patent Ser. No. 12/195,418 on 14 Jan. 2025, and 17584189 which claims the benefit of and priority to U.S. Provisional Application No. 63/141,880 filed 26 Jan. 2021, which are all incorporated herein by reference.

### GOVERNMENT SUPPORT

None

### FIELD OF THE INVENTION

The present invention relates to novel halogenated benzylidene derivatives for the treatment of cancers, specifically in view of promising cancer model data. Specifically, the application is related to a compound the applicants refer to as RNV-125 (Compound 8):

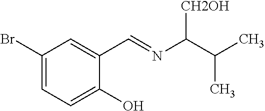 [SMILES: CC(C)C(CO)/N=C/c1cc(Br)ccc1O]

### BACKGROUND OF INVENTION

The applicants have previously discovered and done a considerable amount of research on the HALOGENATED BENZYLIDINE DERIVATIVES of the invention, specifically related to treatments of inflammatory diseases. The applicants have now screened the compound for anti-neoplastic activity using a number of cancer cell-lines.

### SUMMARY OF INVENTION

The present invention relates to the discovery that compounds within a series originally developed as anti-inflammatory agents, including Compound 7 (RNV-124) and Compound 8 (RNV-125), exhibit anti-proliferative (anti-neoplastic) activity against cancer cells. In particular, the invention is directed to the use of RNV-125 as a preferred compound for inhibiting the growth and viability of cancer cells.

In one aspect, the invention provides methods for inhibiting proliferation of cancer cells by contacting the cells with Compound 8 (RNV-125), or a related compound such as Compound 7, in an amount effective to reduce cell viability. The methods are applicable to a variety of cancer types, including breast, lung, skin, colorectal, and prostate cancers.

In another aspect, the invention provides that Compound 8 (RNV-125) exhibits dose-dependent cytotoxic activity in vitro across multiple human cancer cell lines, with activity observed at micromolar concentrations. In certain embodiments, Compound 8 demonstrates half maximal inhibitory concentration (IC50) values in the low micromolar range in selected cancer cell lines, including values below 10 μM and, in some instances, below 5 μM.

In a further aspect, the invention provides pharmaceutical compositions comprising Compound 8 (RNV-125), optionally in combination with Compound 7 or other related compounds, together with one or more pharmaceutically acceptable carriers. Such compositions are useful in the treatment or management of cancer.

The anti-cancer activity of Compound 8 is unexpected in view of its original development as an anti-inflammatory compound, and the demonstrated activity across multiple cancer cell types indicates a broad therapeutic potential.

Describing the invention broadly, the present invention relates to novel Halogenated Benzylidene derivative compounds of the general formula (I)

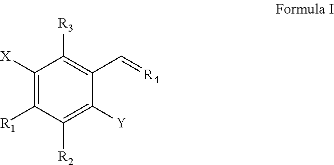

their derivatives, analogues, tautomeric forms, stereoisomers, their polymorphs, pharmaceutically acceptable salts, and pharmaceutically acceptable solvates, wherein X corresponds to any halogen; Y corresponds to hydrogen, any halogen, hydroxyl, alkoxy, nitro, amino or sulphonyl groups; R₁ and R₃ corresponds to any of H, OH or alkyl chain with any number of carbon atoms or modifications therein; R₂ corresponds to a Hydrogen or hydroxyl, a free hydroxyl group or extended chain through an alkyloxy ester or un-substituted or substituted aryloxy ester groups; R₄ corresponds to any naturally occurring or synthesized amino acid, their derivatives like amino acid alcohol and amino acid ester, condensed through their free amino group. Also, R₄ may correspond to an un-substituted or substituted aryl amine, pyridyl amine or amino benzoic acid wherein the free amine group condenses to form a halogenated benzylidene compound. Compounds derived from the saturation of the double bond to R₄ are also included.

More specifically, the present invention relates to a molecule designated by the applicants as “RNV-125”, also referred to as “COMPOUND 8”, having the IUPAC name: 4-Bromo-2-[(1-hydroxymethyl-2-methyl-propylimino)-methyl]-phenol and having the structural formula:

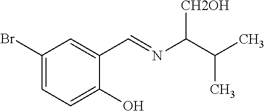 [SMILES: CC(C)C(CO)/N=C/c1cc(Br)ccc1O]

[Note the —CH—₂OH hydroxymethyl group attached to N].

The present invention also relates to a process for the preparation and the therapeutic use of any of said novel compounds or their analogues, their tautomeric forms etc., specifically for treating cancer.

Additionally, the present invention relates to a molecule highly structurally similar to RVN-125, which has been designated by the applicants as “RNV-124” also referred to as “COMPOUND 7”, having the IUPAC name: 2-[(5-Bromo-2-hydroxy-benzylidene)-amino]-3-methyl-butyric acid and having the structural formula:

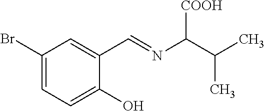 [SMILES: CC(C)C(/N=C/c1cc(Br)ccc1O)C(=O)O]

[Note the —COOH carboxyl group attached to N].

### BRIEF DESCRIPTION OF FIGURES

The original graphs and figures may include colour, which is not shown in this disclosure, but will be made available to the office if requested. The colours are generally represented in the greyscale images by different shades of grey and the meaning of the shading can generally be extrapolated and understood in context.

FIG. 1 shows the effect of Compound-8 (RNV-125) in BT-20 human breast cancer cell line

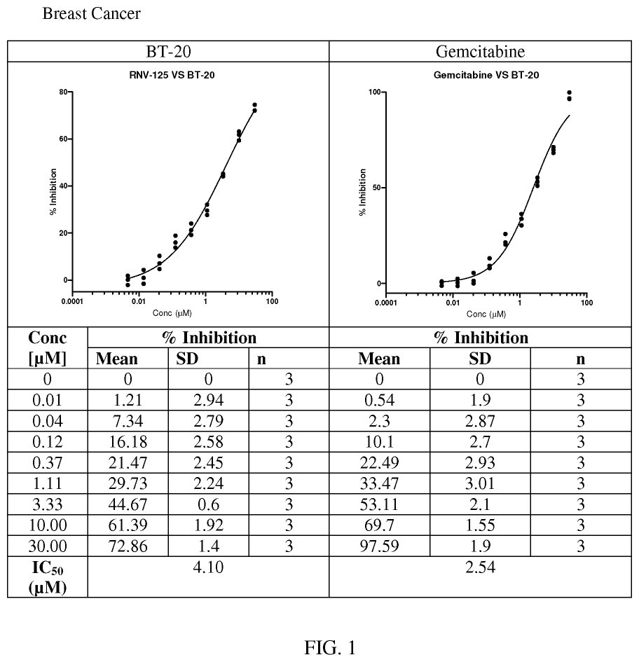

FIG. 2 shows the effect of Compound-8 (RNV-125) in MCF-7 human breast cancer cell line

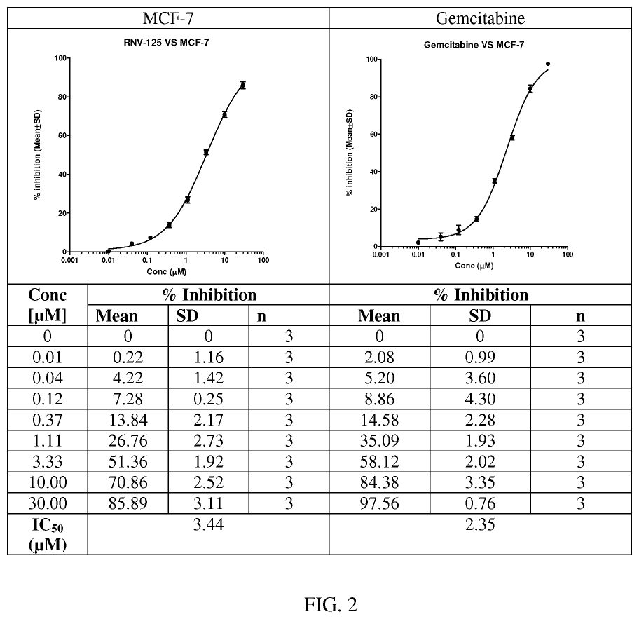

FIG. 3 shows the effect of Compound-8 (RNV-125) in MDA-MB-231 human breast cancer cell line

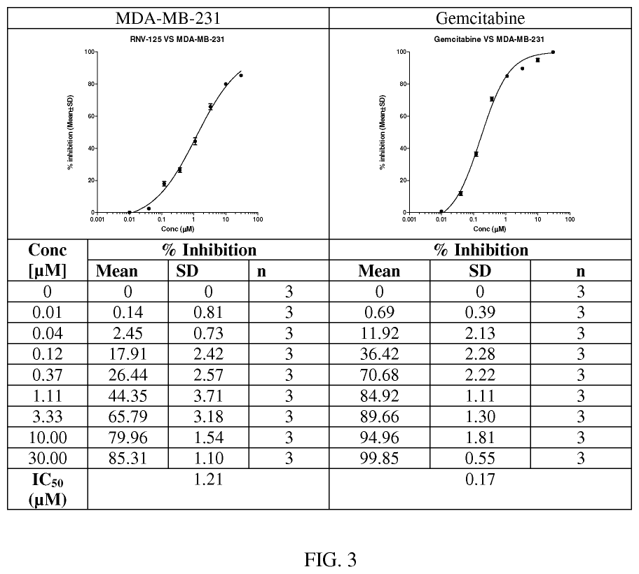

FIG. 4 shows the effect of Compound-8 (RNV-125) in MDA-MB-453 human breast cancer cell line

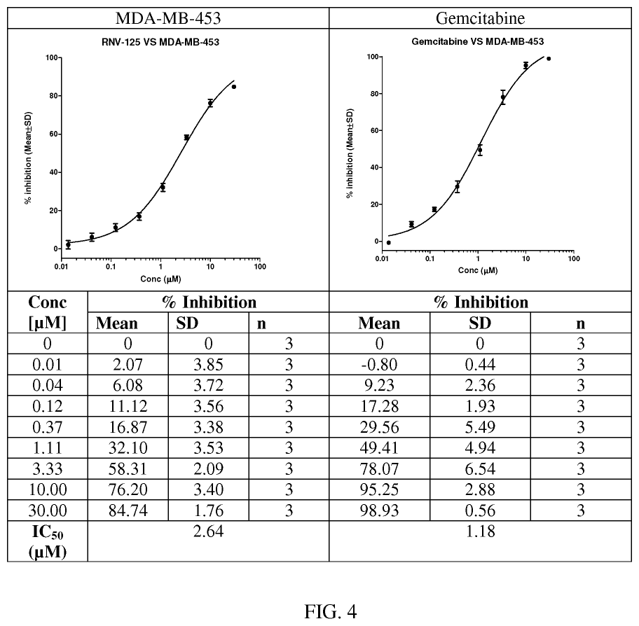

FIG. 5 shows the effect of Compound-8 (RNV-125) in SK-BR-3 human breast cancer cell line

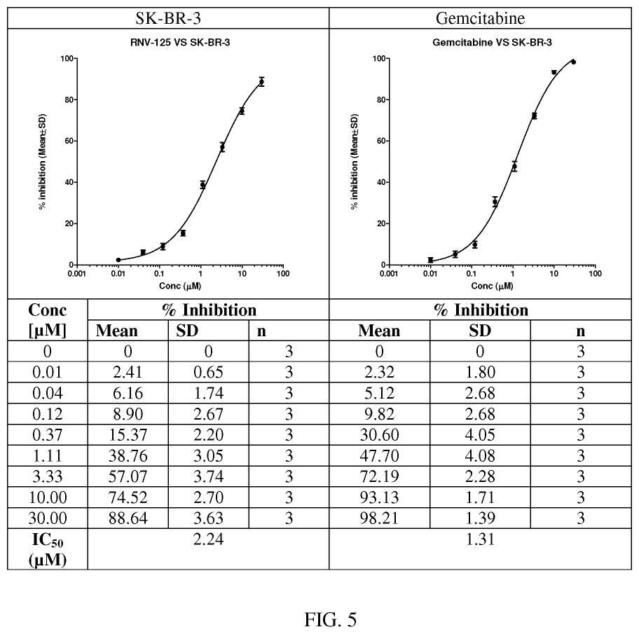

FIG. 6 shows the effect of Compound-8 (RNV-125) in COLO-320 human colorectal cancer cell line

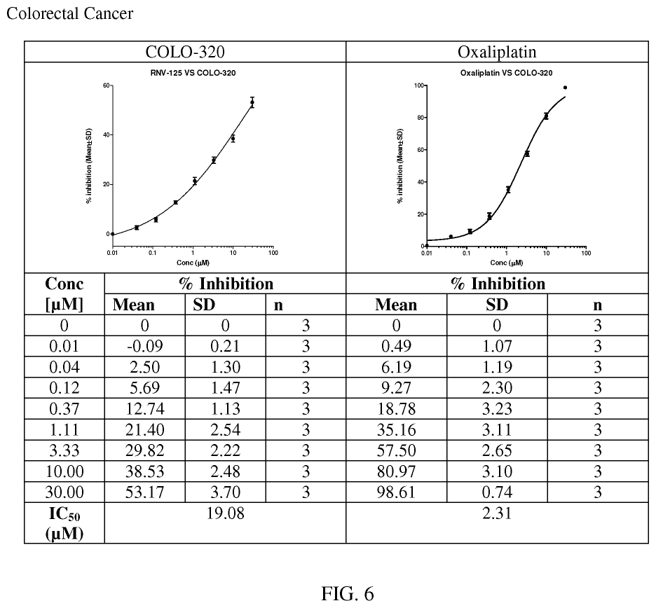

FIG. 7 shows the effect of Compound-8 (RNV-125) in DLD-1 human colorectal cancer cell line

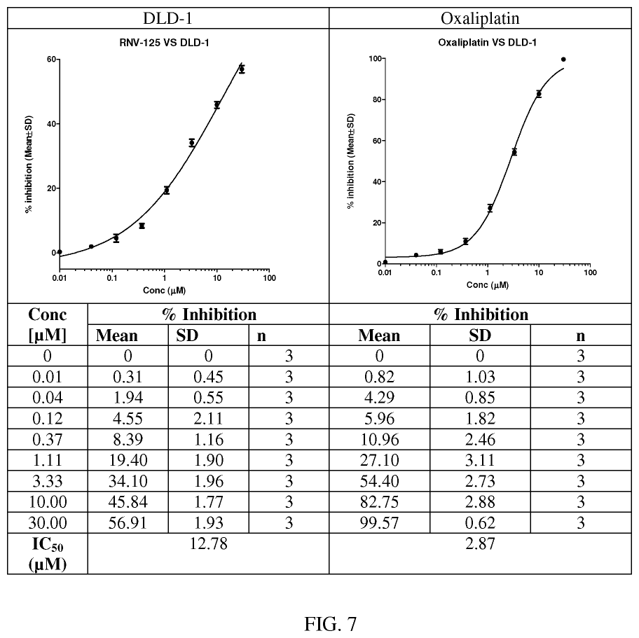

FIG. 8 shows the effect of Compound-8 (RNV-125) in HCC-2998 human colorectal cancer cell line

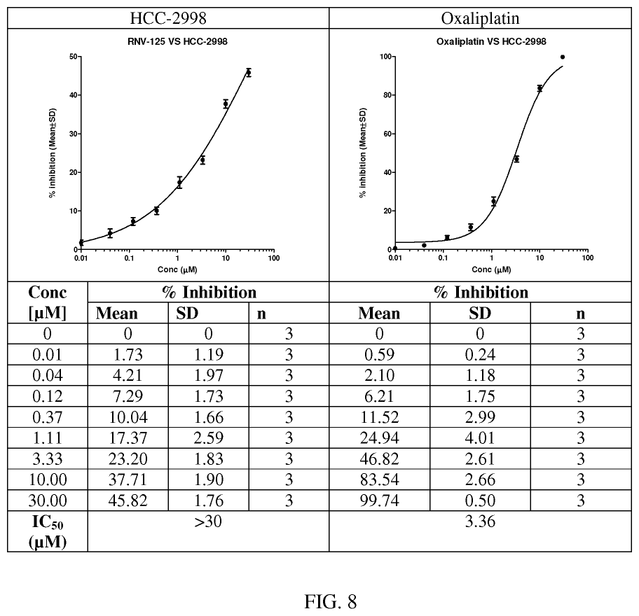

FIG. 9 shows the effect of Compound-8 (RNV-125) in HCT-116 human colorectal cancer cell line

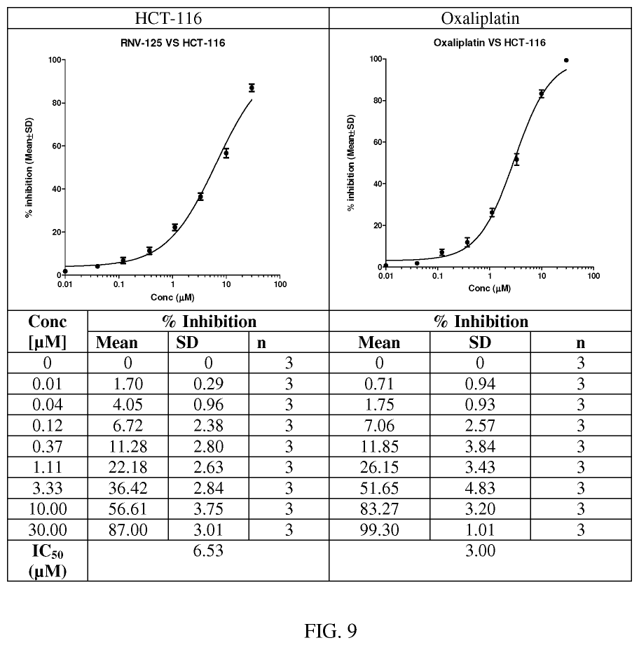

FIG. 10 shows the effect of Compound-8 (RNV-125) in SW-480 human colorectal cancer cell line

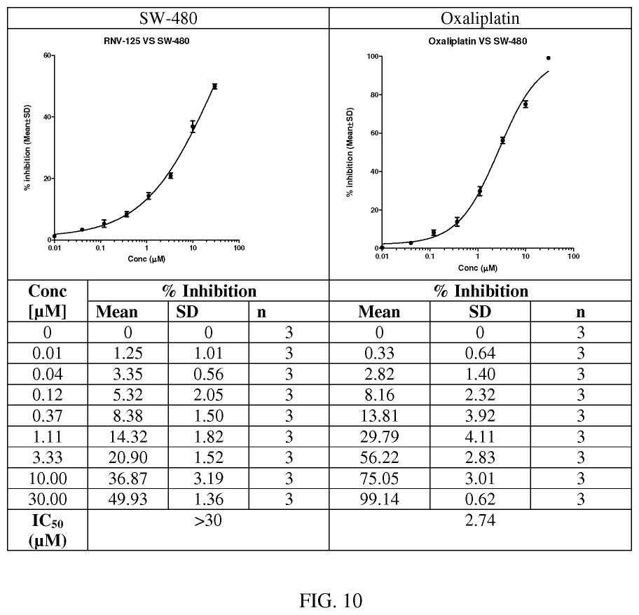

FIG. 11 shows the effect of Compound-8 (RNV-125) in A549 human lung cancer cell line

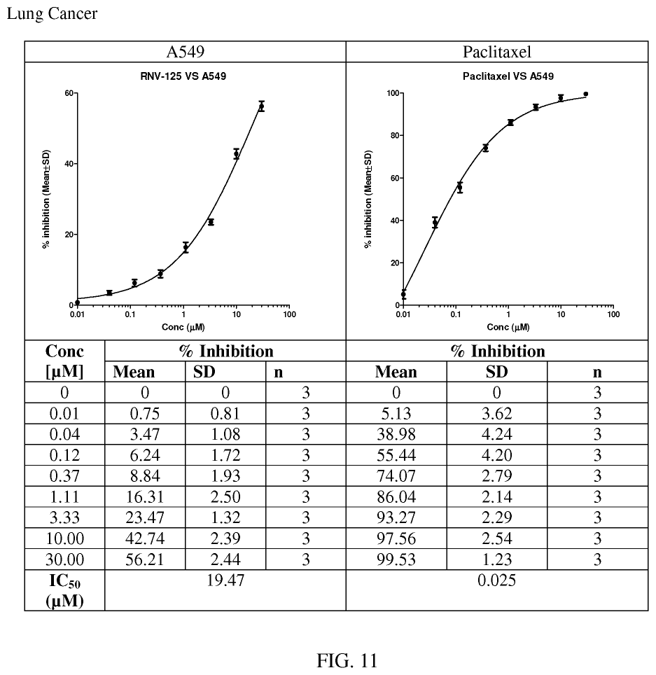

FIG. 12 shows the effect of Compound-8 (RNV-125) in HOP-62 human lung cancer cell line

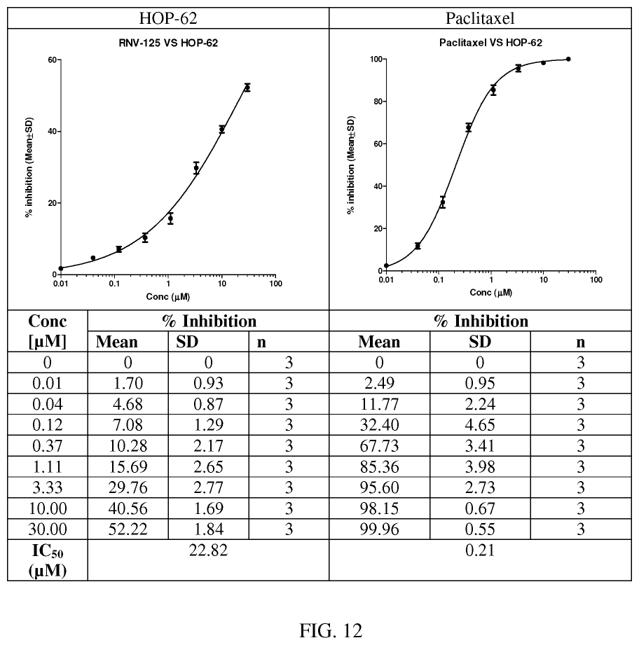

FIG. 13 shows the effect of Compound-8 (RNV-125) in NCI-H82 human lung cancer cell line

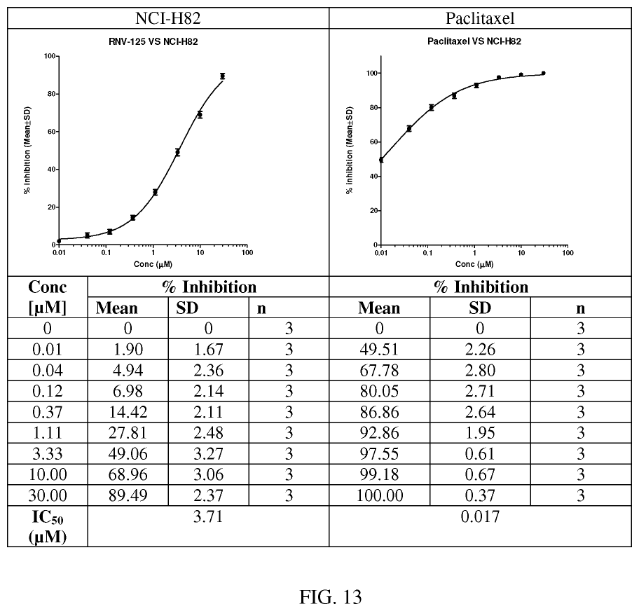

FIG. 14 shows the effect of Compound-8 (RNV-125) in NCI-H460 human lung cancer cell line

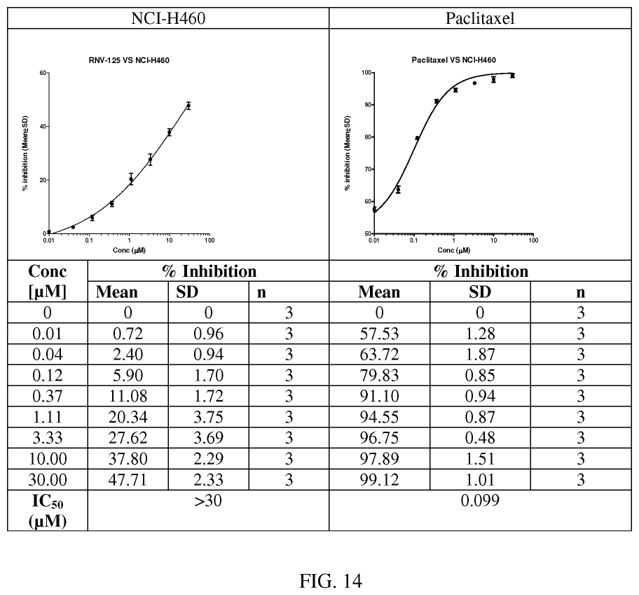

FIG. 15 shows the effect of Compound-8 (RNV-125) in NCI-H1975 human lung cancer cell line

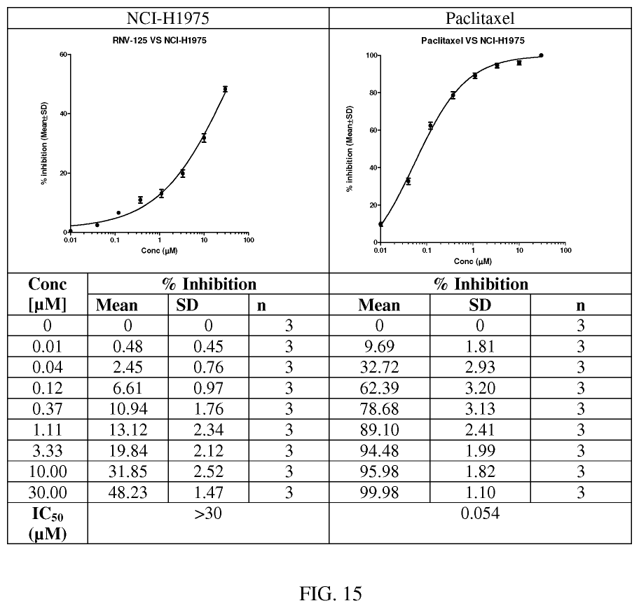

FIG. 16 shows the effect of Compound-8 (RNV-125) in 22RV1 human prostate cancer cell line

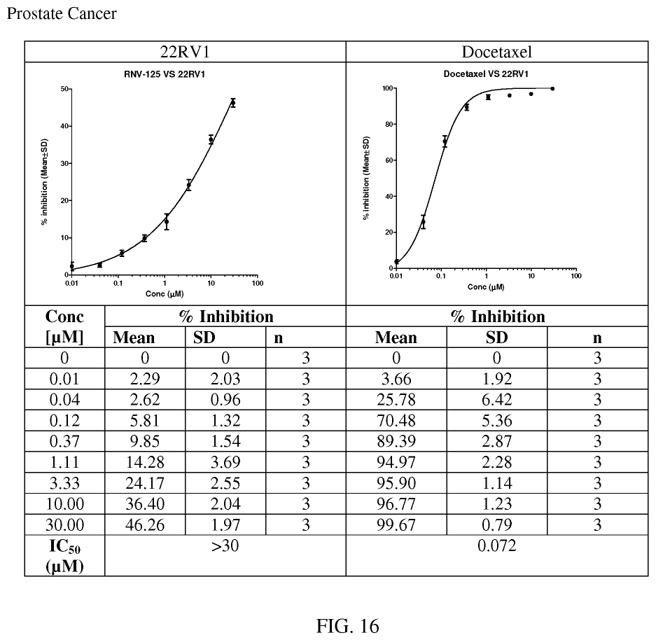

FIG. 17 shows the effect of Compound-8 (RNV-125) in C4-2 human prostate cancer cell line

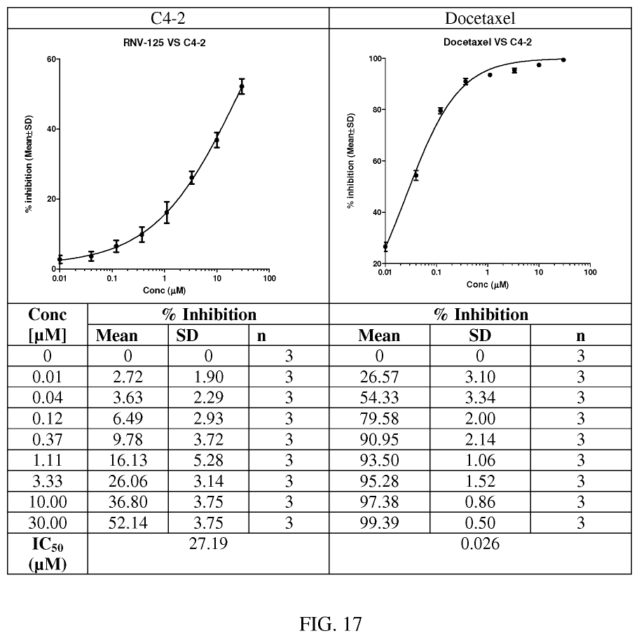

FIG. 18 shows the effect of Compound-8 (RNV-125) in DU-145 human prostate cancer cell line

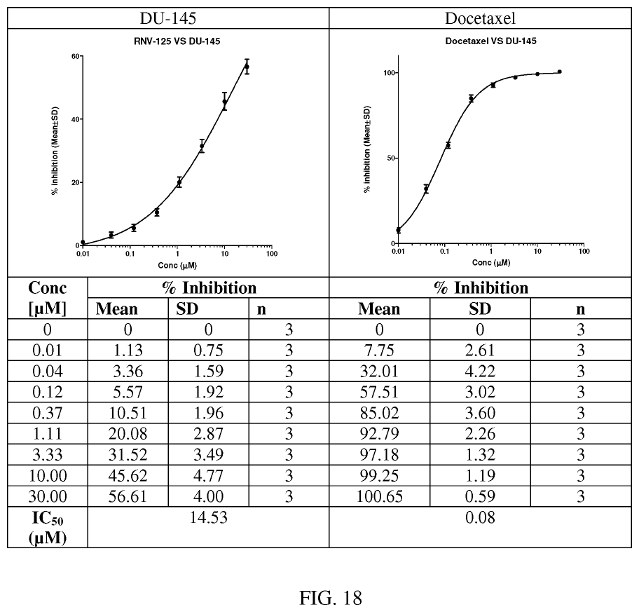

FIG. 19 shows the effect of Compound-8 (RNV-125) in LNCap human prostate cancer cell line

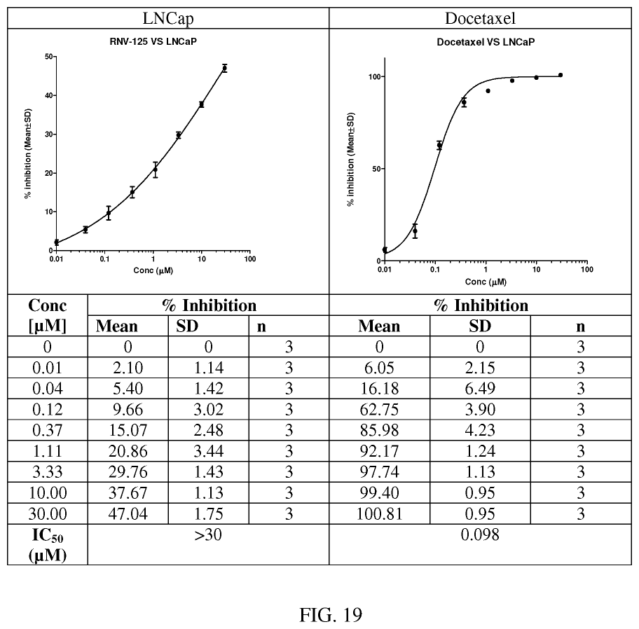

FIG. 20 shows the effect of Compound-8 (RNV-125) in PC-3 human prostate cancer cell line

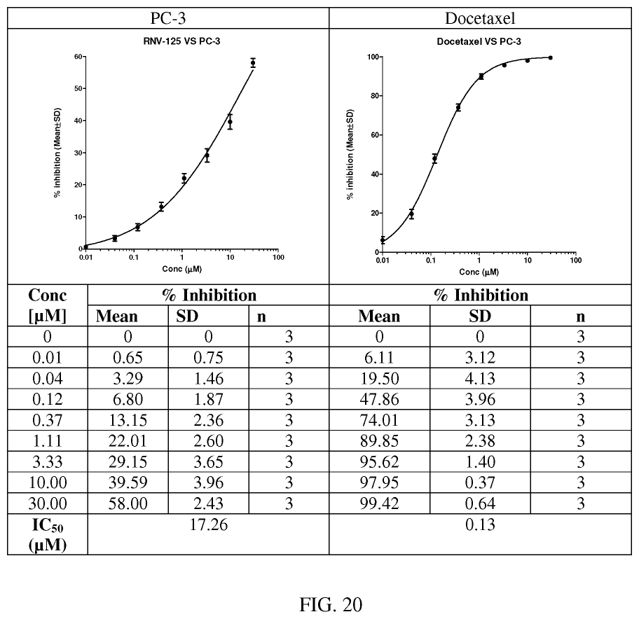

FIG. 21 shows the effect of Compound-8 (RNV-125) in A-375 human skin cancer cell line

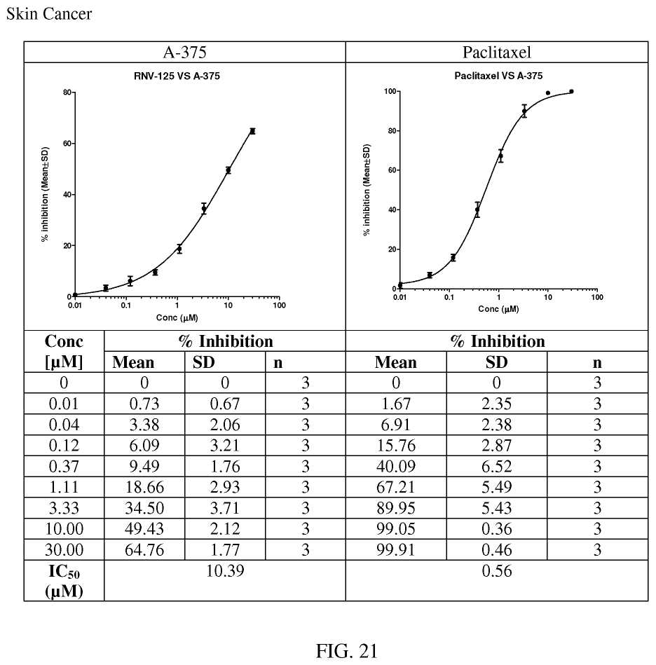

FIG. 22 shows the effect of Compound-8 (RNV-125) in A-431 human skin cancer cell line

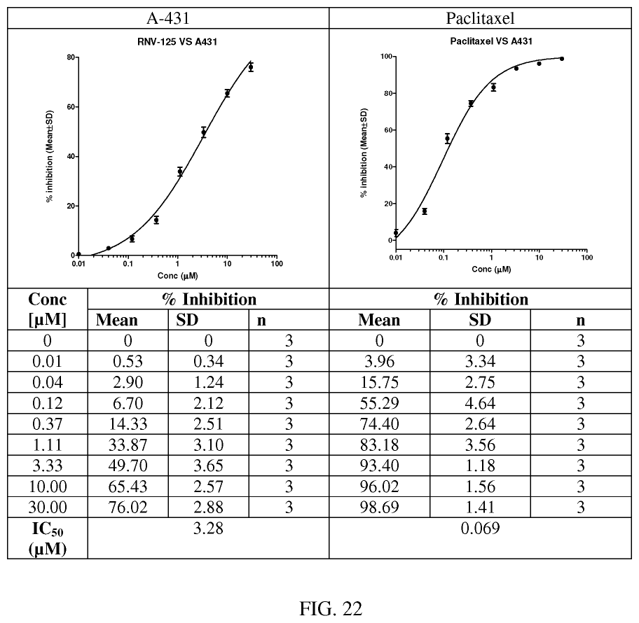

FIG. 23 shows the effect of Compound-8 (RNV-125) in G-361 human skin cancer cell line

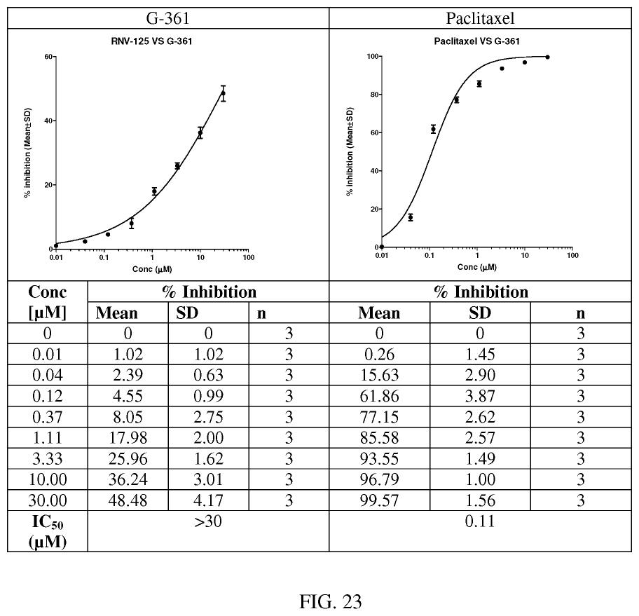

FIG. 24 shows the effect of Compound-8 (RNV-125) in SK-MEL-3 human skin cancer cell line

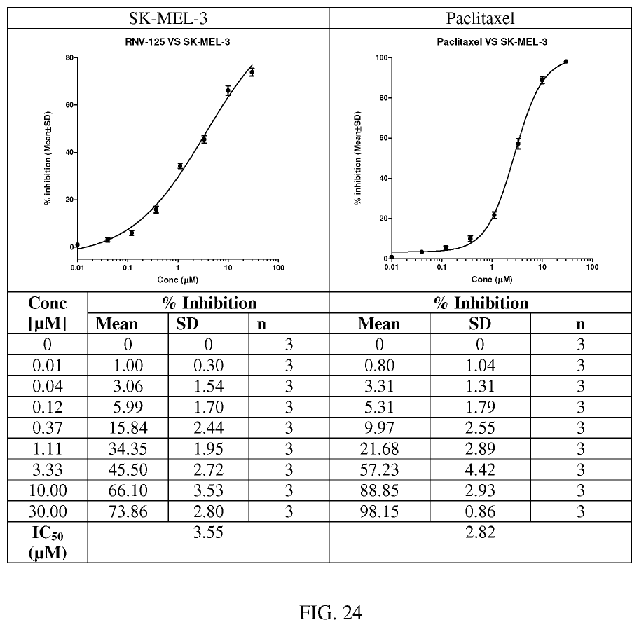

FIG. 25 shows the effect of Compound-8 (RNV-125) in SK-MEL-28 human skin cancer cell line

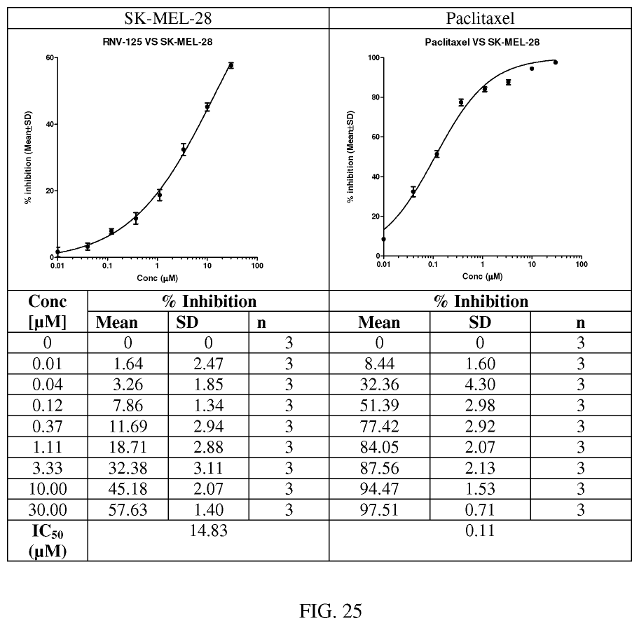

### DETAILED DESCRIPTION OF THE INVENTION

The present invention relates to the discovery that certain compounds originally developed as anti-inflammatory agents, including Compound 7 (RNV-124) and particularly Compound 8 (RNV-125), exhibit anti-neoplastic (anti-proliferative) activity against certain cancer cell lines. In particular, the invention is directed to the identification and use of Compound 8 (RNV-125) as a lead compound demonstrating potent and broad-spectrum inhibition of cancer cell growth.

In one aspect, the invention provides methods for inhibiting proliferation of cancer cells by contacting the cells with a compound selected from Compound 7 and Compound 8 (RNV-125), and particularly Compound 8, for which significant data has been gathered, in an amount effective to reduce cell viability. The invention is supported by in-vitro cytotoxicity data obtained across a panel of human cancer cell lines representing multiple cancer types, including breast, lung, skin, colorectal, and prostate cancers.

The data demonstrate that Compound 8 (RNV-125) produces dose-dependent inhibition of cell viability across multiple cell lines and exhibits greater and more consistent anti-proliferative activity than related compounds within the same chemical series, including Compound 7. In certain embodiments, Compound 8 exhibits inhibitory activity at micromolar concentrations, including concentrations ranging from approximately 0.01 μM to approximately 30 μM.

In further embodiments, Compound 8 demonstrates half maximal inhibitory concentration (IC50) values in the low micromolar range in multiple cancer cell lines.

For example, IC50 values below 10 μM, and in some instances below 5 UM, are observed in selected breast and skin cancer cell lines, indicating significant cytotoxic potency. These results distinguish Compound 8 from other related compounds in the series and establish it as a preferred compound for anti-cancer applications.

The observed anti-cancer activity of Compound 8 is unexpected in view of its original design and development as an anti-inflammatory compound. The ability of Compound 8 to inhibit proliferation across diverse cancer cell lineages suggests a mechanism of action that is distinct from, or broader than, its previously understood pharmacological profile. This cross-lineage anti-proliferative activity supports the use of Compound 8 as a therapeutic or prophylactic agent in oncology.

The cytotoxicity studies underlying the present invention were conducted using cell viability assays in which test compounds were introduced into cell culture media at defined concentrations. The concentrations expressed in micromolar (μM) represent final concentrations in the assay medium following dilution, and reflect exposure of the cancer cells to the compounds under controlled in-vitro conditions.

In further aspects, the invention encompasses pharmaceutical compositions comprising Compound 8 (RNV-125), optionally in combination with Compound 7 or other related compounds, together with one or more pharmaceutically acceptable carriers, excipients, or diluents. Such compositions may be formulated for administration by any suitable route, including oral or parenteral routes, in amounts effective to achieve anti-proliferative effects consistent with the in-vitro activity described herein.

Taken together, the data demonstrate that Compound 8 (RNV-125), within a broader series including Compound 7, possesses previously unrecognized and unexpectedly broad anti-cancer activity, thereby providing a new therapeutic use for these compounds, and particularly Compound 8, in the management and treatment of cancer.

A main embodiment is the compound below used for the treatment of cancers.

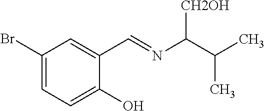 [SMILES: CC(C)C(CO)/N=C/c1cc(Br)ccc1O]

having the following IUPAC name: 4-Bromo-2-[(1-hydroxymethyl-2-methyl-propylimino)-methyl]-phenol and designated by the applicants as “RNV-125”, also referred to as “COMPOUND 8”.

A second embodiment of the present invention relates to a molecule structurally similar to RNV-125, but having a carboxyl group (—COOH) instead of a hydroxymethyl group (—CH₂OH) attached to N. This has been designated by the applicants as RNV-124 also referred to as “COMPOUND 7”, having the IUPAC name: 2-[(5-Bromo-2-hydroxy-benzylidene)-amino]-3-methyl-butyric acid and having the structural formula:

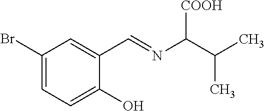 [SMILES: CC(C)C(/N=C/c1cc(Br)ccc1O)C(=O)O]

The present invention also relates to a process for the preparation and the therapeutic use of said novel compounds or their derivatives, analogues, tautomeric forms etc.

RNV-125 is an orally active (and therefore orally deliverable) small molecule derived from natural sources and engineered to enhance certain bioactive and pharmacological activities. The applicants have screened the compound for anti-neoplastic activity using a number of cancer cell-lines, and this data is shown in the drawings.

RNV-125 shows strong anti-neoplastic activity as shown in the drawings. RNV-125 appears to be a novel molecule for the treatment of cancers using oral administration.

Compound 8 (RNV-125) was originally designed for use as an inflammatory agent. The applicants unexpectedly found that Compound 8 (RNV-125) also significant antineoplastic activity, which makes it an unexpected lead molecule for the development of a new class of orally active antineoplastic pharmaceuticals.

The applicants have systematically carried out a number of in vitro assays using cancer cell lines, and present a selection of evidence herein, specifically in the drawings.

The applicants disclose a method for treating cancer, the method comprising administering to a patient suffering from cancer, a pharmaceutical formulation comprising Compound 8 (RNV-125), administered by oral route, wherein said formulation comprises Compound 8 (RNV-125) in a pharmaceutically acceptable formulation.

An in-vitro cytotoxicity study of RNV-125 was undertaken against 25 cancer cell lines by MTT assay.

Serial dilutions of eight different concentrations (30, 10, 3.333, 1.111, 0.370, 0.123, 0.041, 0.014 μM) were used for the compound.

The cell viability assay was done in the following cell lines of these cancer types:
Breast Cancer cell lines: MDA-MB-231, MDA-MB-453, MCF-7, SK-BR-3, BT-20;
Lung Cancer cell lines: A549, NCI-H1975, HOP-62, NCI-H82, NCI-H460;
Skin Cancer cell lines: A-375, A-431, G-361, SK-MEL-28, SK-MEL-3;
Colon Cancer cell lines: HCT-116, DLD-1, HCC-2998, COLO320, SW-480;
Prostate Cancer cell lines: DU-145, PC3, LNCaP, 22Rv1, C4-2;

The figures and the associated tables provided herewith are from in-vitro cell-based studies. The table shows the concentrations of the Compound 8 (RNV-125) used. Serial dilutions of eight different concentrations (30, 10, 3.333, 1.111, 0.370, 0.123, 0.041, 0.014 μM) of Compound 8 (RNV-125) were used in the study.

Compounds master stock were prepared in DMSO at 10 mM conc. Serial dilutions of eight different concentrations were prepared for each compound. Compounds were initially be diluted 3.3× in 100% DMSO and then further diluted 5× (20% DMSO) in complete media. 10 μl of this 5× dilution was then added to the assay plate containing 190 μL media to achieve a final concentration of 30 μM and below (1% DMSO).

Compound 8 (RNV-125) was originally designed for use as an inflammatory agent, but the applicants have unexpectedly found that Compound 8 (RNV-125) also provides significant anti-cancer activity.

### Formulations of the Compounds of the Invention

The formula of the compounds of the invention include (but are not limited to) those listed below.

One key molecule of the invention is “RNV-125”, also referred to as “COMPOUND 8”, having the IUPAC name:
4-Bromo-2-[(1-hydroxymethyl-2-methyl-propylimino)-methyl]-phenol; and having the structural formula:

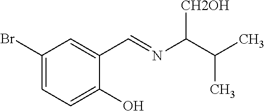 [SMILES: CC(C)C(CO)/N=C/c1cc(Br)ccc1O]

The present invention also relates to a process for the preparation and the therapeutic use of any of said novel compounds or their analogues, their tautomeric forms etc.

Another molecule of the invention is very structurally similar to RNV-125, and has been designated by the applicants as RNV-124 also referred to as “COMPOUND 7”, having the IUPAC name: 2-[(5-Bromo-2-hydroxy-benzylidene)-amino]-3-methyl-butyric acid (Compound 7); and having the structural formula:

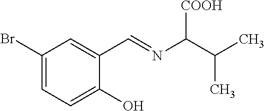 [SMILES: CC(C)C(/N=C/c1cc(Br)ccc1O)C(=O)O]

The invention encompasses derivatives, pharmaceutically acceptable salts, esters, solvates, hydrates, prodrugs, and polymorphs thereof, including all stereoisomeric forms (e.g., enantiomers, diastereomers), tautomers, and isotopically enriched forms of the compounds. Unless otherwise specified, such references encompass both racemic mixtures and individual enantiomers or diastereomers. Derivatives of the compounds include compounds structurally modified by substitution, elimination, or addition of one or more chemical groups, provided such derivatives retain the essential pharmacological activity. Preferred salts for the compounds listed above are hydrochloride, hydrobromide, sodium, potassium, or magnesium.

According to another feature of this present invention, the invention provides a process for the preparation of the compound represented by the formula I, wherein all symbols are as defined as earlier, as shown in Scheme 1.

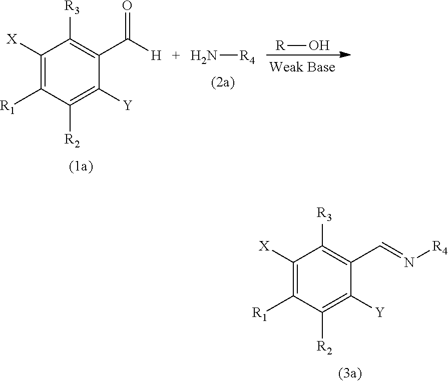

The reaction of a compound of general formula (1a) with a compound of general formula (2a) to produce a compound of the general formula (3a) may be carried out in an inert atmosphere which may be maintained by using inert gases such as nitrogen, argon or helium. The reaction may be carried out in a polar protic solvent like alcohols, preferably methanol or ethanol and in the presence of weak bases like DEA, TEA, Isopropylamine, pyridine, pipridine and the like, but more preferably with a base like TEA.

The temperature of the reaction may range between 40 to 80° C., optimally between 60 to 80° C. and the duration may extend between 1 to 10 hours. The schiff base thus formed may be precipitated or could be extracted after suitable workup procedures such as water quenching. The resultant molecule is the halogenated benzylidine derivative of general formula (3a) where the groups have been defined earlier.

The invention is explained in detail in the EXAMPLES given below which are provided by way of illustration only and therefore should not be construed to limit the scope of the invention.

### Example 1

### Synthesis of 2-[(5-Bromo-2-hydroxy-benzylidene)-amino]-3-methyl-butyric acid (“COMPOUND 7”)

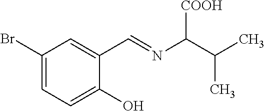 [SMILES: CC(C)C(/N=C/c1cc(Br)ccc1O)C(=O)O]

### Step (i)

### Synthesis of Valine Methyl Ester Hydrochloride

Valine (50.0 g) was taken in a clean and dry round bottom flask and methanol (150 ml) was added. Thionyl chloride (34.50 ml) was introduced in to the reaction mixture and it was refluxed at 70-80° C. for 6 hours with constant stirring. The excess solvent was then removed by distillation and the solid product obtained was stored under nitrogen. Yield—75.0 g

### Step (ii)

Valine methyl ester hydrochloride (75.0 g) and 5-bromo salicaldehyde (50.0 g) were taken in a clean and dry round bottom flask and methanol (250 ml) was added with constant stirring. Triethylamine (50 ml) was introduced in to the mixture and it was refluxed for 8 hours at 65-70° C. Molecular sieves were also added to scavenge the water produced during the reaction. The reaction mass was then dissolved in acetone (200 ml) and filtered to remove the undissolved material. A solid precipitate was obtained when water (500 ml) was added to the filtrate. This was then filtered and dried. Yield—54.6 g.

Step (iii)

2-[(5-Bromo-2-hydroxy-benzylidene)-amino]-3-methyl-butyric acid methyl (9.0 g) was taken in a clean and dry round bottom flask and NaOH solution (3.69 g/120 ml) was added to it with constant stirring. Acetone (90 ml) was added and the mixture was maintained at room temperature for 3 hours. The reaction mixture was then cooled to 25° C. and the pH was brought to 5.5 using 1:1 HCl solution. The precipitated solid was filtered, washed with water followed by hexane and dried. Yield 5.80 g. NMR—10.21 (1H, s), 8.13 (1H, s), 6.65-7.62 (Aromatic), 5.0 (1H, s), 3.89 (1H, d), 2.04 (1H, m), 1.12 (6H, d).

### Example 2

### Synthesis of 2-[(5-Bromo-2-hydroxy-benzylidene)-amino]-3-methyl-butyric acid (“COMPOUND 8”)

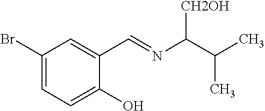 [SMILES: CC(C)C(CO)/N=C/c1cc(Br)ccc1O]

### Step (i)

Synthesis of valinol. Valine (11.7 g) was taken in a clean and dry round bottom flask and tetrahydrofuran (100 ml) was added with constant stirring. Boron trifluoride ethyl ether complex 14.2 ml) and borane dimethylsulphide (10 ml) were added and the reaction mixture was refluxed at 40-50° C. for 4 hours. The reaction mixture was acidified with 0.5N HCl and the THF layer was separated and washed twice with water. The solvent was then dried over sodium sulphate and evaporated to obtain the product. Yield 8.0 g.

### Step (ii)

Valinol (10 g) and 5-bromo salicaldehyde (4.0 g) were taken in a clean and dry round bottom flask and methanol (100 ml) was added with constant stirring. TEA (1.0 ml) was added and the mixture was refluxed for 4 hours at 65-70° C. The excess solvent was then removed by distillation and the crude product was dissolved in water (150 ml). Chloroform was used to extract the product from the solution and the separated organic layer was dried over sodium sulphate and then removed under reduced pressure to obtain the final product. Yield—5.2 g. NMR—6.65-7.62 (Aromatic), 5.0 (1H, s), 3.89 (1H, d), 2.04 (1H, m), 1.12 (6H, d).

1. A method for inhibiting proliferation of cancer cells, the method comprising contacting the cancer cells with a pharmaceutical formulation in an amount effective to reduce cell viability, wherein the pharmaceutical formulation comprises a compound RNV-125 (Compound 8), having the IUPAC name 4-Bromo-2-[(1-hydroxymethyl-2-methyl-propylimino)-methyl]-phenol, and having the following formula:

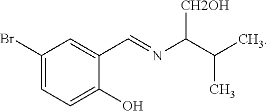 [SMILES: CC(C)C(CO)/N=C/c1cc(Br)ccc1O]

2. The method of claim 1, wherein the cancer cells are human cancer cells.

3. The method of claim 1, wherein the cancer cells are selected from breast cancer, lung cancer, skin cancer, colorectal cancer, and prostate cancer cell lines.

4. The method of claim 1, wherein the compound is present at a concentration of from about 0.01 μM to about 30 μM.

5. The method of claim 1, wherein contacting the cancer cells with said pharmaceutical formulation results in at least 30% inhibition of cell viability.

6. The method of claim 5, wherein the inhibition of cell viability is at least 50%.

7. The method of claim 1, wherein said pharmaceutical formulation exhibits an IC50 of less than 20 μM in at least one human cancer cell line.

8. The method of claim 7, wherein the IC50 is less than 10 μM.

9. The method of claim 8, wherein the IC50 is less than 5 μM.

10. The method of claim 9, wherein the IC50 is below 5 μM, in breast and skin cancer cell lines.

11. The method of claim 1, wherein the cancer cells comprise a breast cancer cell line selected from MDA-MB-231, MCF-7, MDA-MB-453, BT-20, and SK-BR-3, and wherein Compound 8 (RNV-125) exhibits an IC50 of less than 5 μM.

11. The method of claim 1, wherein the cancer cells comprise a colorectal cancer cell line selected from HCT-116, DLD-1, COLO-320, HCC-2998, and SW-480, and wherein Compound 8 (RNV-125) exhibits an IC50 of less than 20 μM.

12. A pharmaceutical formulation comprising a RNV-125 (Compound 8), having the IUPAC name 4-Bromo-2-[(1-hydroxymethyl-2-methyl-propylimino)-methyl]-phenol, and having the following formula: 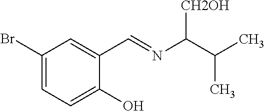 [SMILES: CC(C)C(CO)/N=C/c1cc(Br)ccc1O]
and a pharmaceutically acceptable carrier, formulated to inhibit the proliferation of cancer cells.

13. A method of inhibiting proliferation of human breast cancer cells, the method comprising contacting a human breast cancer cell line selected from MDA-MB-231, MCF-7, MDA-MB-453, BT-20, and SK-BR-3 with Compound 8 (RNV-125) having the IUPAC name 4-Bromo-2-[(1-hydroxymethyl-2-methyl-propylimino)-methyl]-phenol, and having the following formula: 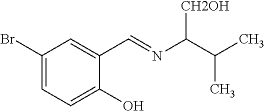 [SMILES: CC(C)C(CO)/N=C/c1cc(Br)ccc1O]
at a concentration of from 0.01 μM to about 30 μM, wherein Compound 8 (RNV-125) exhibits an IC50 of less than 5 μM in said cell line.

14. The method of claim 13, wherein the human breast cancer cell line is MDA-MB-231, and wherein Compound 8 (RNV-125) exhibits an IC50 of less than 2 μM in said cell line.
